(19) United States
(12) Patent Application Publication
(10) Pub. No.: US 2018/0116513 A1
(43) Pub. Date: May 3, 2018

(54) WIRE-FREE MONITORING DEVICE FOR ACQUIRING, PROCESSING AND TRANSMITTING PHYSIOLOGICAL SIGNALS

(71) Applicants: Ravi BHOGU, Telangana (IN); Monitra Healthcare Private Limited, Telangana (IN)

(72) Inventor: RAVI BHOGU, TELANGANA (IN)

(73) Assignee: Monitra Healthcare Private Limited, Telangana (IN)

(21) Appl. No.: 15/572,432

(22) PCT Filed: May 11, 2016
(86) PCT No.: PCT/IB2016/052694

§ 371 Date: Nov. 7, 2017

(30) Foreign Application Priority Data
May 12, 2015 (IN) 2418/CHE/2015

(51) Int. Cl. A61B 5/00; A61B 5/0408; A61B 5/04; A61B 5/0404; A61B 5/0478; A61B 5/0488; A61B 5/0492; A61B 5/0496

(52) U.S. Cl. CPC A61B 5/0006; A61B 2562/0223; A61B 5/04085; A61B 5/684; A61B 5/04017; A61B 5/0404; A61B 5/7267; A61B 5/002; A61B 5/0478; A61B 5/04884; A61B 5/04886; A61B 5/0492; A61B 5/0496; A61B 2560/0412; A61B 2562/0209; A61B 2562/0219; A61B 5/04087

(57) ABSTRACT

Embodiments of the present disclosure relate to an electrode device to acquire, process and transmit physiological signals. The device eliminates the need for inconvenient wires attached at different locations on patient's body. The device comprises a sensor patch comprising plurality of sensors/electrodes, configured to be in contact with a skin surface of a human body, to measure physiological of the patient. Also, the, device comprises a wire-free module,, embedded in the sensor patch, comprising an auto-orientation module to detect the orientation of the sensor patch on the human body using a plurality of sensors. The wire-free module comprises a processing module to process one or more signals received from the plurality of sensors/electrodes. Further, the wire-free module comprises a detection module to detect one or more processed signals as a predefined physiological signal and a transmission module to transmit the detected physiological signal to a mobile device.

### TECHNICAL FIELD

Embodiments of the present disclosure are related, in general to acquiring physiological signals, but exclusively relate to a wire-free electrode device for acquiring electrocardiogram (ECG) signals.

### BACKGROUND

An electrocardiogram (ECG) interpretation has been the basis of diagnosis of cardiovascular disease, since the inception of ECG. However, the administration of ECG testing has been limited largely to diagnostic clinics, hospitals, emergency rooms and recently to remote cardiac monitoring devices. Although the benefit of remote cardiac monitoring device is significant, patient compliance remains a big challenge and these devices are prone to lead placement errors.

Currently, there are solutions for ECG diagnostics, such as, resting ECG performed at diagnostic clinics and remote or ambulatory monitoring devices such as 24-72 hour Holter monitors, event loop monitors and ECG transmission to smartphones. The Holter technology includes a smaller recorder with flashcard or memory to record and store data from 2 to 3 ECG leads attached to a patient's chest. The data is collected continuously for a period range of 24 hours to 48 hours, and analyzed in digital format. To increase the correlation between detected heart rhythm abnormalities and symptoms, a dairy is incorporated for making manual entry of the symptoms a patient is having on a regular basis. The recorders use patient-activated event markers or annotations, specified for the time of day. The major advantages of Holter monitoring are the ability to continuously record ECG data and the lack of need for patient participation in the transmission of data. However, the short duration of monitoring may be inadequate if symptoms are infrequent.

The available Holter monitors have a recording capability of with up to two weeks. However, Holter monitoring have limitations such as frequent noncompliance with wearing the device during to discomfort from wires around the body along with keeping a log of symptoms and using event markers, which significantly limits the diagnostic value of these devices. Also, the Holter monitoring does not have real-time data analysis, which is an important clinical limitation of these devices.

Further, there are intermittent patient recorders or event activated recorders, also referred to as event monitors for monitoring ECG of patient. Continuous looping monitors are attached to the patient through chest electrodes or a wrist band and the data is recorded. This is performed only when the patient activates. Also, these devices have automatic triggers that recognize slow, fast, or irregular heart rates. Once activated, data is stored for a programmable fixed amount of time before the activation or looping memory and a period of time after the activation. These devices are referred as external loop recorders (ELRs). There is another less sophisticated form of event monitor is the post event recorder, which is not worn continuously i.e. non-looping, but instead is applied directly to the chest area of a patient, once a symptom develops. Hence, these devices do not have storage unit to allow recording of the rhythm, before the device is activated. Generally, event monitors are used for a period ranging from 14 to 30 days monitoring period. The data recorded is transmitted trans-telephonically to a central monitoring station and uploaded to a personal computer for analysis.

The ELRs are better than Holter monitors because they are of smaller size, allow ECG monitoring for longer time periods, and may provide nearly real-time data analysis when the patient transmits a recording in proximity to the symptomatic event. However, a significant percentage of patients are noncompliant with continuous application of the ELRs, mostly because of discomfort from wires around the body, lead irritation/poor skin contact during exercise due to long term usage.

The continuous and post event recorders require a degree of technological sophistication to transmit the stored data trans-telephonically to the central monitoring station. The technical equivalent of this skill is the ability to use an automatic bank teller machine. One of the prior art showed that 84.5% of patients were able to perform a test transmission, but a successful recording and diagnostic transmission was performed by only 58.9% of patients. Patients living alone were much less likely to use an ELR effectively, and factors such as worry about or fear of symptoms and their impact on quality of life were associated with successful use of the devices.

Another known device, allows automatic transmission of triggered events over the cellular network which has no requirement for the patient to transmit the data. For the devices which need not be worn continuously, post event recorders such as wristbands or handheld devices that need to be applied to the chest at the time of symptoms, the initiation of the arrhythmia that may provide a clue to the arrhythmic mechanism is missed, and short arrhythmias that terminate before the device is applied will not be recorded.

Real-time continuous attended cardiac monitoring systems represent the newest form of external ambulatory monitors developed to combine the benefits and to overcome the limitations of Holter monitors and standard ELRs. They are worn continuously and are similar in size to the standard ELR. They automatically record and transmit arrhythmic event data from ambulatory patients to an attended monitoring station. The data can also be recorded through patient-triggered activation, which is referred as mobile or real-time cardiac telemetry systems (MCOT).

The common areas of noncompliance with ambulatory monitoring include the unwillingness to wear a device continuously, intolerance of the electrodes because of rash, failure to activate a monitor in association with symptoms, and inability to trans-telephonically download the information. A study shows that only 53% of patients wore a device and provided recordings five days a month during the entire 6 month monitoring period. Failure to activate a device in association with symptoms is a significant problem with monitoring with Holter and standard event recorders without automatic triggers. Also, in another study using loop recorders to diagnose syncope, despite patient education and test transmissions, 23% of patients who had recurrence of their syncopal symptoms failed to activate their loop recorder properly.

Further, there were several incidents and serious events, including deaths reported, which are associated with remote cardiac monitoring. The most frequently cited types of failure in remote cardiac monitoring reports are communication issues; delayed or incorrect placement and power failures, which includes disconnection of devices from their power sources and failure to replace batteries.

Accordingly, a need exists for a device for monitoring multiple ECG signals without wires to improve patient compliance, which also automatically recognizes the device orientation thereby capturing ECG signals correctly without user intervention to eliminate incorrect lead placement errors, which also records, and monitors ECG signals in real time. Also, the device has reduced size, cost, and making it wireless, less complex, portable and easy to use.

### SUMMARY

The shortcomings of the prior art are overcome and additional advantages are provided through the provision of method of the present disclosure.

Additional features and advantages are realized through the techniques of the present disclosure. Other embodiments and aspects of the disclosure are described in detail herein and are considered a part of the claimed disclosure.

In an aspect of the present disclosure, a wireless physiological device for acquiring and processing physiological signals is provided. The device comprises a sensor patch comprising plurality of sensors/electrodes, configured to be in contact with a skin surface of a human body, to measure physiological of the patient. Also, the device comprises at least one wire-free module, embedded in the sensor patch, comprising an auto-orientation module to detect the orientation of the sensor patch on the human body using a plurality of sensors. The wire-free module also comprises a processing module to process one or more signals received from the plurality of sensors/electrodes. Further, the wireless module comprises a detection module to detect one or more processed signals as a predefined physiological signal and a transmission module to transmit the detected physiological signal to a mobile device.

Another aspect of the present disclosure is a method of acquiring and processing physiological signals using a wireless monitoring device. The method comprises identifying orientation of a sensor patch placed on a human body. Next, processing one or more signals received from a plurality of sensors/electrodes configured in the sensor patch. Further, detecting a physiological signal from the processed signals and transmitting the detected physiological signal to a mobile device.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE ACCOMPANYING DRAWINGS

The accompanying drawings, which are incorporated in and constitute a part of this disclosure, illustrate exemplary embodiments and, together with the description, serve to explain the disclosed principles. In the figures, the left-most digit(s) of a reference number identifies the figure in which the reference number first appears. The same numbers are used throughout the figures to reference like features and components. Some embodiments of device or system and/or methods in accordance with embodiments of the present subject matter are now described, by way of example only, and with reference to the accompanying figures, in which:

FIG. 1 illustrates a block diagram of an exemplary wire-free electrode for acquiring ECG signals in accordance with an embodiment of the present disclosure;

FIG. 2A shows an illustration of wire-free electrode with plurality of sensors in accordance with an embodiment of the present disclosure;

FIG. 2B shows an illustration of wire-free electrode with four sensors in accordance with an alternate embodiment of the present disclosure;

FIG. 2C shows an illustration of wire-free electrode with four sensors in accordance with another alternative embodiment of the present disclosure;

FIG. 2D shows an illustration of wire-free electrode with six sensors in accordance with an alternate embodiment of the present disclosure;

FIG. 2E shows an illustration the wire-free electrode placed on a human body, in accordance with an example embodiment of the present disclosure;

FIG. 2F shows an illustration of multiple ECG signals recorded to describe the electrical activity of the heart, in accordance with another embodiment of the present disclosure;

FIG. 3 illustrates an exemplary block diagram of a signal processing module, in accordance with an embodiment of the present disclosure;

FIG. 4 illustrates a wireless channel ECG system, in accordance with an embodiment of the present disclosure; and

FIG. 5 illustrates a flowchart showing optimal machine learning by the wire-free system based on the resources available, in accordance with an embodiment of the present disclosure.

### DETAILED DESCRIPTION

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

While the disclosure is susceptible to various modifications and alternative forms, specific embodiment thereof has been shown by way of example in the drawings and will be described in detail below. It should be understood, however that it is not intended to limit the disclosure to the particular forms disclosed, but on the contrary, the disclosure is to cover all modifications, equivalents, and alternative falling within the spirit and the scope of the disclosure.

The terms “comprises”, “comprising”, or any other variations thereof, are intended to cover a non-exclusive inclusion, such that a setup, device or method that comprises a list of components or steps does not include only those components or steps but may include other components or steps not expressly listed or inherent to such setup or device or method. In other words, one or more elements in a device or system or apparatus proceeded by “comprises . . . a” does not, without more constraints, preclude the existence of other elements or additional elements in the device or system or apparatus.

In the following detailed description of the embodiments of the disclosure, reference is made to the accompanying drawings that form a part hereof, and in which are shown by way of illustration specific embodiments in which the disclosure may be practiced. These embodiments are described in sufficient detail to enable those skilled in the art to practice the disclosure, and it is to be understood that other embodiments may be utilized and that changes may be made without departing from the scope of the present disclosure. The following description is, therefore, not to be taken in a limiting sense.

An exemplary embodiment of the present disclosure is a device to acquire and physiological signals. The device is also referred as a system or a wire-free electrode.

The device eliminates the need for inconvenient wires attached at different locations on patient's body. In one embodiment, the device performs real-time cardiac event detection and automated data transmission from single wire-free/wireless electrode. The system comprises an arrangement of an electrode that allows acquisition of several cardiac bio-potentials or ECG signals.

Another embodiment of the present disclosure is a method of acquiring physiological signals. The method is performed by automated recognition of electrode orientation which facilitates determination of plurality of leads. The real-time detection classifies the sensed signals by the leads as one of critical events and non-critical events. Another embodiment is reducing patient noncompliance, which may be one of the biggest challenges for both short and long term remote cardiac monitoring, and minimize care giver/user errors involved in placement of traditional ECG leads that create artifacts, mimic pathologies, and hinder proper ECG interpretation.

In an aspect of the present disclosure, a wireless physiological device for acquiring and processing physiological signals is provided. The device comprises a sensor patch comprising plurality of sensors/electrodes, configured to be in contact with a skin surface of a human body, to measure physiological signals of the patient. The physiological signal is at least one of electrocardiogram (ECG), electroencephalogram (EEG), electromyogram (EMG), Electroretinogram (ERG), Electrooculography (EOG), Electroolfactogram (EOG), Electropalatogram (EPG), Electrogastroenterogram (EGEG), Electrocochleography (ECOG), Galvanic skin response (GSR) and any other physiological signal. Also, the device comprises at least one wire-free module embedded in the sensor patch, comprising an auto-orientation module to detect the orientation of the sensor patch on the human body using a plurality of sensors. The wire-free module is also referred as wireless module. The wire-free module comprises a processing module to process one or more signals received from the plurality of sensors/electrodes. Further, the wire-free module comprises a detection module to detect one or more processed signals as a predefined physiological signal and a transmission module to transmit the detected physiological signal to a mobile device.

Another aspect of the present disclosure is a method of acquiring, processing and transmitting physiological signals using a wireless monitoring device. The method comprises identifying orientation of a sensor patch placed on a human body. Next, processing one or more signals received from a plurality of sensors/electrodes configured in the sensor patch. Further, detecting a physiological signal from the processed signals and transmitting the detected physiological signal to a mobile device and thereafter to the server on the internet or cloud. In addition, the machine learning is done at either the sensor, mobile device or server based on the available connectivity and bandwidth.

In one embodiment of the present disclosure a remote cardiac monitoring device for monitoring electrocardiogram (ECG) signals remotely, is disclosed. The remote cardiac monitoring device is also referred as a cardiac system or wire-free cardiac device or wireless cardiac device or wireless cardiac electrode or wire-free cardiac electrode or wireless electrode or wire-free electrode. In one embodiment, the device is a single electrode. The device improves frequent patient noncompliance due to discomfort from wires around the body by making a device without wires and at the same time capture electrocardiogram signals. The device requires no effort from the user/care giver in affixing a single electrode on a part of body of the user without any requirement to fix the device in any particular orientation or in multiple places as in traditional devices, wherein the user may be a patient. The device comprising a new arrangement of a cardiac electrode that allows acquisition of several cardiac bio-potentials and a method for automated recognition of electrode orientation which facilitates determination of up-to 6 lead electrocardiogram equivalent to Lead I, Lead II, Lead III, aVR, aVL and aVF. The cardiac device would also remove any caregiver/user errors involved in placement of traditional electrocardiogram leads that create artifacts, mimic pathologies, and hinder proper electrocardiogram interpretation.

In one embodiment, the wire-free electrode comprises an on-board intelligence which facilitates real-time detection of cardiac events by classifying the events into at least one of critical and non-critical cardiac. The critical cardiac events have the highest priority in data transmission and alerting the care-giver or the doctor or medical practitioner, so that, an appropriate intervention is accorded and the situation is treated on a priority basis.

The cardiac electrode activates a monitor in association with symptoms of a patient, to keep a log of symptoms. Also, the cardiac electrode use event markers and trans-telephonically transmits the information. The complete cardiac system comprises a wire-free cardiac electrode, application module on a mobile device and at least one remote monitoring server. The at least one remote monitoring server stores the data from the cardiac electrode and performs analytics on the data based on the requirement. In an example, a patient may perform at least one of pressing of a button on the electrode to inform the system about a symptomatic event and logging or recording the event information along with the associated symptoms on the mobile device application module. The system is recognizes the type of data network such as, but not limited to, Wi-Fi, 2G, 3G, 4G, voice network and any other data network. Thereafter, the system manages data transmission according to significance allotted to cardiac events based on one of critical and non-critical nature. In one embodiment, the system by default may attempt to transmit ECG data via the lowest cost available data network and the critical events shall be transmitted on priority over any of the available network.

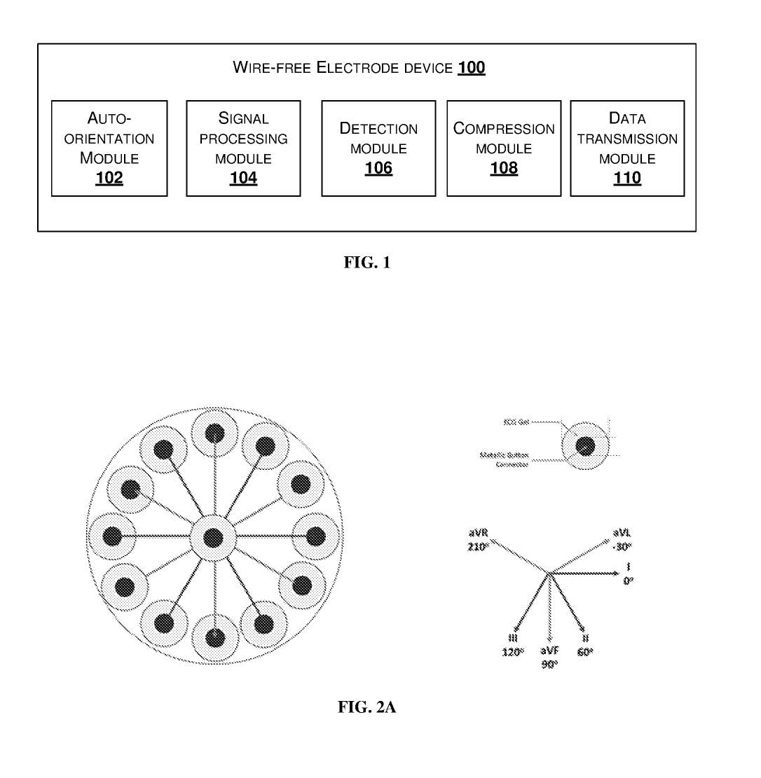

FIG. 1 illustrates a block diagram of an exemplary wire-free electrode for acquiring physiological signals, in accordance with some embodiments of the present disclosure. The wire-free electrode is also referred as a wireless physiological device or wireless physiological monitoring device or wire-free cardiac electrode or remote cardiac monitoring device or cardiac system or wireless cardiac device or wireless cardiac electrode or wire-free cardiac electrode or wireless electrode or wire-free electrode. The physiological signals acquired are at least one of electrocardiogram (ECG), Electroencephalography (EEG), motion, airflow of respiratory system, body temperature, arterial oxygen saturation level, blood pressure, electromyogram (EMG), Electroretinogram (ERG), Electrooculography (EOG), Electroolfactogram (EOG), Electropalatogram (EPG), Electrogastroenterogram (EGEG), Electrocochleography (ECOG), Galvanic skin response (GSR) and any other physiological signal.

As shown in FIG. 1, the wire-free electrode 100 comprises an auto-orientation module 102, signal acquisition and processing module 104, detection module 106, compression module 108 and data transmission module 110.

The device 100 is configured such that, a user or a patient may use the device with ease. Also, the device does not require the patient to orient the device in a particular angle on the predefined part of the human body. The device may be peeled like any other body worn bands/medical devices and pasted on to a predefined of the part human body. When the device is placed on the predefined part of the human body, the device self-orients itself to know the precise location of plurality of micro-sensors, configured in the device, in the X & Y plane. This self-orientation or auto-orientation is performed by the auto-orientation module 102 is also referred as an auto-orientation engine.

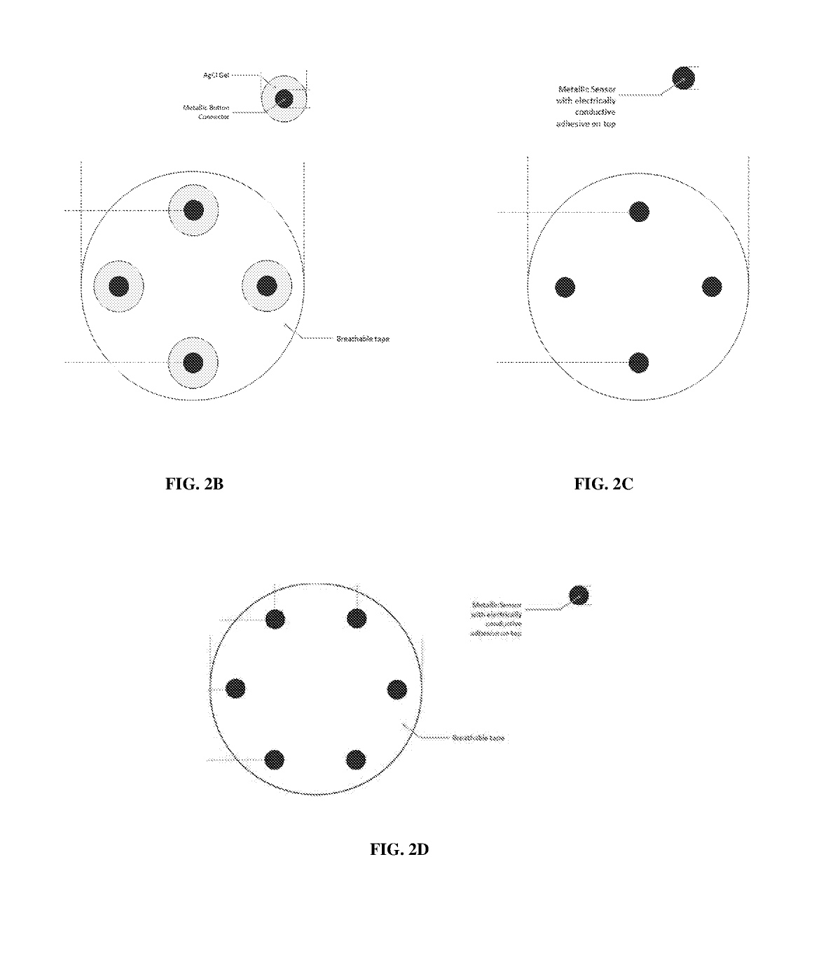

The wire-free electrode device 100 also comprises plurality of sensors as shown in FIG. 2A, they perform at least one of self-recognition of electrodes orientation, real-time cardiac event detection, effortless logging of symptomatic events with associated markers and automated data transmission based on nature of captured events. The device provides the user/patient ease of use and at the same time provides significant diagnostic information from physician viewpoint for effective correct diagnosis and proper therapy in real time.

As shown in FIG. 2A, the cardiac electrode is automated that performs recognition of ECG signals from the electrode orientation and facilitates determination of about six ECG leads. A user or patient may perform at least one of pressing a button on the electrode to inform the device/system about a symptomatic event and logging/recording an event along with the associated symptoms on a mobile device application module. The ECG leads are equivalent to Lead I, Lead II, Lead III, aVR, aVL and aVF from 12 lead ECG, in one embodiment.

FIG. 2B shows an illustration of wire-free electrode having four sensors/electrodes in accordance with an alternate embodiment of the present disclosure. As shown in FIG. 2B, the wire-free electrode uses Ag/AgCl gel for each sensor.

FIG. 2C shows an illustration of wire-free electrode with four sensors/electrodes in accordance with another alternative embodiment of the present disclosure. As shown in FIG. 2C, the wire-free electrode uses conductive adhesive on the metallic sensor, for each sensor.

FIG. 2D shows an illustration of wire-free electrode with six sensors, in accordance with an alternate embodiment of the present disclosure. As shown in FIG. 2D, the wire-free electrode uses conductive adhesive on the metallic sensor, for each sensor/electrode in one embodiment. The sensor/electrode use Ag/AgCl gel, in another embodiment of the present disclosure.

The automatic orientation of the device is performed by a plurality of sensors configured in the auto-orientation module 102. Each of the plurality of sensors is one of accelerometer, gyroscope and magnetometer, which facilitate measurement of orientation of the patch in the X & Y axis. The plurality of sensors is calibrated and any change in the orientation of the device 100 once placed on the human body, as shown in FIG. 2E, is recognized and the device analyzes the changed orientation to find the location of the sensors in the X&Y plane of the human body. Based on the orientation of plurality of sensors, the device picks up the appropriate bio-potential signals from the portion of the human body, for calculation of ECG limb leads. Similarly, multiple ECG leads may be recorded to describe the electrical activity of the heart adequately. FIG. 2F illustrates the ECG lead patterns that are obtained through the plurality of sensors placed configured in the device, these are equivalent leads to the standard ECG lead configuration derived without the use of wires/cables causing patient discomfort in medium-to-long term monitoring.

As shown in FIG. 2A Leads I, II, and III is represented schematically in terms of a triangle, called Einthoven's triangle. The ECG comprises only the recordings from leads I, II, and III. The Einthoven's triangle shows the spatial orientation of the three standard limb leads (I, II, and III). In one embodiment, lead I points horizontally, left pole (LA) is positive and its right pole (RA) is negative. Therefore, lead I=LA−RA, Lead II points diagonally downward, lower pole (LL) is positive and upper pole (RA) is negative. Therefore, lead II=LL−RA. The Lead III points diagonally downward. Its lower pole (LL) is positive and its upper pole (LA) is negative. Therefore, lead III=LL−LA.

In one embodiment, Lead I+Lead III=Lead II. The voltage in lead I to that in lead III facilitate to provide voltage in the Lead II.

In one embodiment, the wire-free electrode use different types of sensors as shown in FIGS. 2A-2D. The sensors may either be in contact or non-contact media. The contact sensors may have one of wet interfaces, dry interfaces and any other form of services.

The wet contact sensor comprises a small metal plate surrounded by an adhesive fabric, which is coated with a conducting wet gel to aid transmission of the signal. The sensor is assembled with an electrolyte gel in which the principle anion is Cl-. The Cl- is an attractive anion for sensors/electrode applications, since the skin interface contains an excess of chloride ions in solution or perspiration. A silver chloride is very slightly soluble in water, so most of the silver chloride precipitates out of the solution onto the silver sensors/electrode and contributes to a silver chloride deposit. The sensors are converted from metallic Ag to Ag/AgCl by electrolytic or chemical conversion processes. The metal plate can be replaced by any other conductive materials such as conductive carbon fiber loaded ABS plastic.

In one embodiment, the dry sensors comprises of plates made of metals such as, but not limited to silver, stainless steel, brass, and nickel; conductive carbon nanotubes or any other conductive material. A conductive adhesive is used to transfer the bio-potential signals from the surface of the skin to the sensors. The non-contact sensors comprise active electronic circuits to capacitive pick up the bio-potential signals.

Referring back to FIG. 1, the signal acquisition and processing module 104 performs the processing of the signals received from the auto-orientation module 102. The signal acquisition and processing module 104 also referred as a signal acquisition and processing engine or signal processing module or pre-processing module or acquisition module. The processing module 104 comprises a front-end processing module for differentiating between a noise and the desired signal or predefined signal, received from the plurality of sensors configured in the auto-orientation module 102, which is of very small amplitude. The predefined signal is the ECG signal. The front-end processing module comprises at least one instrumentation amplifier configured to reduce the common mode signal. In one embodiment, the instrumentation amplifier operates on +/−3V and used for the large input voltage range. Also, the processing module 104 comprises operational amplifiers for signal conditioning for the ECG signals. The signal chain for the ECG acquisition system consists of instrumentation amplifiers, filters implemented through op-amps, and ADCs.

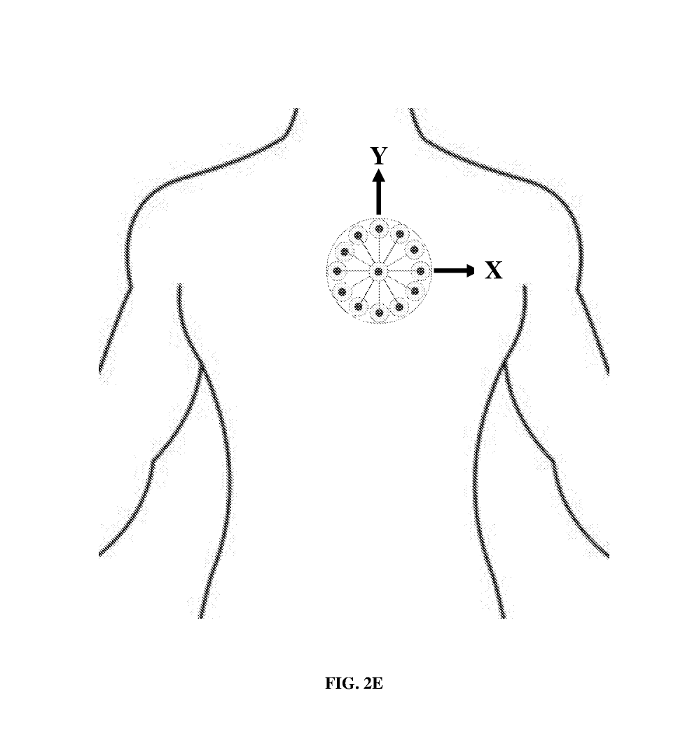

FIG. 3 illustrates an exemplary block diagram of a signal processing block or module 104, in accordance with some embodiments of the present disclosure. The signal processing module is also referred as signal acquisition and pre-processing module or engine. The signal processing module is critical as actual sensed signal value may be about 0.5 mV in an offset environment of 300 mV. The other factors such as, but not limited to AC power supply interference, RF interference from surgery equipment, and implanted devices such as pace makers and physiological monitoring systems may also impact accuracy of sensed signal value. The sources of noise in ECG is at least one of baseline wander that is a low frequency noise, power line interference about 50 Hz or 60 Hz noise from power lines, muscle noise that is very difficult to remove as it is in the same region as the actual signal and other interferences such as radio frequency noise from other equipment.

The interference is a common mode noise across both terminals of the differential amplifier. The interference is removed by at least one of isolating the analog front-end 202 ground electronics from the digital system, using one or more instrumentation amplifiers with very high common mode rejection ratios, and driving the patient body with an inverted common mode signal. User's or Patient's one sensor-node may be considered as reference and driven with a signal which is the inverted average of multiple available ECG channels to reduce the common mode interference; Shielding the device to prevent high frequency RF from being coupled into the system. The aim in the design of the front-end is to minimize the noise which is coupled into the system.

In one embodiment, baseline wander is a low frequency component present in the ECG system, which is due to offset voltages in the sensors/electrodes, respiration, and body movement. Also, an offset limits a maximum value of gain which may be obtained from the instrumentation amplifiers. At higher gains, the signal may saturate and the noise is removed using a high pass filter 204. One of the specifications of the ECG is the input referred noise which should be less than 30 uV for the entire system at 150 Hz bandwidth. In one embodiment, a high resolution Analog to Digital Converter (ADC) 206 is configured in the processing module 104, a single stage of gain achieved by the instrumentation amplifiers. The hardware-based high pass filter is removed, and the baseline wander is carried over into the digital domain. The filtering process performed in the digital domain is cost effective.

In one embodiment, a control unit or a microcontroller is configured into the system, which reduces the overall cost of the wire-free cardiac device or system. FIG. 3 illustrated the signal flow chain for implementing the system without the hardware high pass filter. In this case, the digital filter block may implement effective filtering after the signal is acquired by the ADC, and thereby the complexity of the front-end is reduced significantly.

According to the IEC specification, the bandwidth of the ECG required is from 0.5 Hz to 150 Hz. The cardiac device should have a mechanism to detect pacemakers, which may be detected by having one of hardware and application module. If the detection is performed by an application module, the sampling rate of the ADC must be of the order of 3 to 4 KSps. The advantage of having the application module for pacemakers is that changes in firmware may adapt the ECG machine to different kinds of pacemakers. In most of the high frequency noise may be filtered before it is sampled by the ADC. The device is shielded to prevent high frequency radiated noise from being coupled. Once the data is sampled by the ADC, a digital FIR filter having the desired cutoff frequency is implemented, which removes high frequency noises in the ECG trace. The amplitude of power line noise is very huge and generally gets coupled into the system despite care to prevent common mode noise in the digital domain. Power line noise is removed by implementing a notch filter at 50/60 Hz in the digital domain.

Referring back to FIG. 1, the detection module 106 performs the detection of the sensor signals. Upon detection of the signals, the classification of one of electrocardiogram (ECG), electroencephalogram (EEG), electromyogram (EMG), Electroretinogram (ERG), Electrooculography (EOG), Electroolfactogram (EOG), Electropalatogram (EPG), Electrogastroenterogram (EGEG), Electrocochleography (ECOG), Galvanic skin response (GSR) and any other physiological signal is performed into different disease categories is a complex pattern recognition task, in one embodiment. The ECG signals are classified with high accuracy and the device offers a potential of an affordable mass screening for cardiac abnormalities. After the classification of the sensed signals by finding the characteristic shapes of the ECG that discriminate effectively between the required diagnostic categories. In one embodiment, the detected signals, also referred as datasets, are used for heart diseases involve different features.

The data storage and compression module 108 receives the data from the detection module and stores the data in the internal memory or database. The data storage and compression module 108 is also referred as a compression module or compression engine. In one embodiment, the data from sensors may be stored on a flash memory configured in the sensor patch and may be retrieved using USB cable. The compression module 108 uses a lossless compression method to ensure the subtle changes in ECG signals are preserved in the original form and do not introduce any artifacts that may distort clinical diagnosis. As, the ECG signal comprises repetition of the basic morphologies of signal consisting of P, QRS & T waves. The substantial portions of the ECG signal involve minimal changes in amplitude called isoelectric baseline except for noise, P, QRS & T waves. The duration of the isoelectric baseline is inversely proportional to the heart rate. At normal heart rate range of 60 to 100 bpm, the duration of the isoelectric baselines is quite long. As amplitude changes are minimal at the isoelectric baseline, this portion of the signal requires significantly less number of bits and thereby enabling high compression.

The ECG data compression techniques are limited to the amount of time required for compression and reconstruction, the noise embedded in the raw ECG signal, and the need for accurate reconstruction of the P, Q, R, S, and T waves.

The data transmission module 110 is configured in the data transmission engine resides in the gateway and is divided in to three parts, a network manager, event manager and data request manager. The data transmission module 110 is also referred as data transmission engine or transmission module. The transmission module uses a data transmission protocol which is at least one of Bluetooth, Wireless fidelity (Wi-Fi), second generation (2G), third generation (3G), long term evolution (LTE) and any other wireless protocol. A network manager configured in this module 110 determines the wireless network characteristics such as, but not limited to availability, signal strength, bandwidth and call drops. Also, the network manager is responsible for transmission of bio-potential data such as ECG, cardio-thoracic impedance, body motion along with the events as prioritized by the event manager. The event manager is configured in data transmission module 110, to list the events based on the level of priority determined by the detection module 110. In a case where real-time bandwidth not available on the wireless network, the event manger will buffer the events with highest priority first and then order the remaining depending on the level of priority and timestamp.

In one embodiment, a data request manger is configured in the data transmission module 110, to handle requests from the server for performing at least one of changing clinician parameters for the machine learning module present on the gateway and the sensors status of the electronic components on the sensor and gateway along with the performance statistics which allows the system to understand the wear-tear-life of components on the field itself and change from automated transmission of all data such as, but not limited to real-time or transmission of data on-demand or transmission of events based on the machine learning systems on the sensor and/or gateway. The data manager block to perform machine learning from the physiological data to obtain one of critical and non-critical event. the transmission module transmits physiological data using at least one of minimum available bandwidth data network for non-critical events and any available data network for critical events.

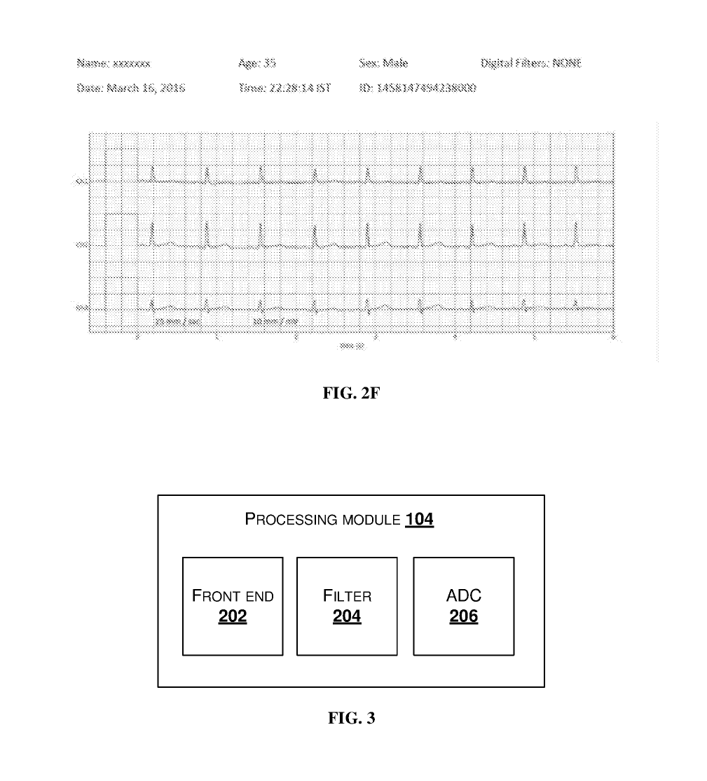

FIG. 4 illustrates a wireless channel ECG system, in accordance with an embodiment of the present disclosure. In one embodiment, the ECG system is used for detecting heart rhythm disorder events, based on the availability of the wireless networks. The system for detecting heart rhythm disorder comprises two wireless channels; one wireless channel is one of Bluetooth Classic, Smart, Zigbee, Wi-Fi and other forms of channels between sensors and a Gateway, which could be a mobile phone, as shown in FIG. 4. The second channel is one of Wi-Fi, a mobile network such as, but not limited to, 2G, 3G, 4G or other advanced RF networks, between the Gateway and a backend server at the cardiac monitoring center, as shown in FIG. 4.

Also, the system acquires the ECG signals in real-time and performs data analysis of multiple lead electrocardiogram. The electrode comprises on-board intelligence which facilitates real-time detection of cardiac events by classifying critical versus non-critical cardiac ones, which is as shown in the FIG. 1. The critical cardiac events will be given highest order of preference in data transmission and alerting the care-giver so that appropriate intervention can be accorded and the situation is treated on a priority basis.

Further, the failure of patient to activate a monitor in association with symptoms, to keep a log of symptoms and use event markers and inability to trans-telephonically transmit the information significantly limits the diagnostic value of these devices. The system is an integrated platform consisting of the wire-free cardiac electrode, application module which is configured in a portable or a handheld device such as, but not limited to a mobile phone, laptop, tablet and PDA. FIG. 2 shows an application module and a server configuration associated with the wireless cardiac electrode in accordance with an embodiment of the present disclosure. As shown in FIG. 2, the application module comprises an event log module, database module also referred as knowledge platform, data manager and a display. The event log module records time stamp or time record of the ECG signals being acquired. Also the event log module is facilitated with an audio recording option for additional information or symptoms of a user whose ECG signals are acquired. The database module also referred as data manager synchronizes data associated with the ECG signals with a remote server. The display also referred as a display manager displays the information associated with the acquired ECG signals such as, but not limited to historical data, real time acquired data and any other information of the user. Also, the display provides graphical user interface (GUI) for a user to input data.

The remote monitoring server stores the data or information received from the wire-free cardiac single electrode, in the storage unit and performs analytics or analysis as on need basis. The remote server comprises a web-server module for communication or connectivity with a mobile device and wire-free cardiac electrode; an information security module for providing data security which is HIPPA compliance and provides information audit logs; a data analytics module for performing offline ECG analysis or analytics, aggregate data and provide summary about risk stratification and trend analysis; and at least one database for storing the data associated with the acquired ECG signals, opinion by an expert on the analyzed data, data analytics information.

One embodiment of the present disclosure is machine learning of the wire-free electrode for pattern recognition. For example, the cardiac events may be time critical from a therapeutic standpoint and require the highest emergency in case of certain critical events, in that scenario the device has intelligence that automatically decides the place where appropriate level of machine learning takes place based on the availability of wireless channels. The real-time machine learning will always reside on the place where the highest computing power resides when the wireless channels are available.

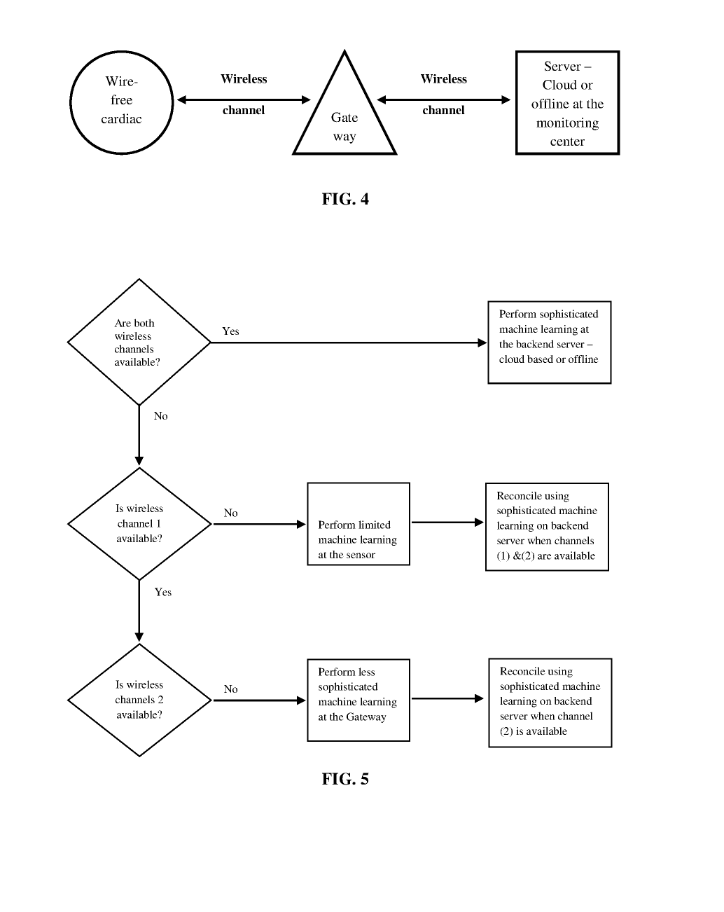

FIG. 5 illustrates a flowchart showing optimal machine learning by the wire-free system based on the resources available, in accordance with an embodiment of the present disclosure. As shown in the FIG. 5, at the sensor level the system recognizes R waves and performs RR interval analysis. Depending on the heart rate and the RR interval changes, events are identified and given priority. In one embodiment, at the gateway level the system recognizes the presence of a predefined disease conditions such as, but not limited to, atrial fibrillation and prioritize them over normal ones. At the backend server, the system is configured to determine all statistics and heart rhythm conditions such as, but not limited to, sinus tachycardia—number of episodes, duration, average rate, range; Bradycardia—number of episodes, duration, average rate, range; Pauses—number of episodes, duration, range; Junction rhythms or ectopy—burden (%), quantity; Atrio ventricular block (type I, type II 2:1, high-grade)—quantity; Complete heart block (third-degree)—quantity, duration; Atrialectopy—burden (%), quantity; Atrial fibrillation—burden (%), range, rate, average; Atrial flutter—burden (%), range, rate, average; Supra ventricular ectopy or tachycardia—burden (%), quantity; Wide complex tachycardia—quantity, rate; Ventricular ectopy (single, couplet, triplet, bigeminy, trigeminy)—type, burden (%), quantity; Ventricular tachycardia (≥3 beats)—sustained (≥30 s) or non-sustained (<30 s), burden (%).

The system incorporates the patient data and physician feedback into machine learning. Each event classified by the system will be corroborated by physician interpretation to make the system's learning stronger with time. The system should be able to detect cardiac events early in the life-cycle and determine the efficacy of therapeutic intervention provided by the physician.

Embodiments of the present disclosure relates to a method of acquiring and processing physiological signals using a wireless monitoring device. The method comprises identifying orientation of a sensor patch placed on a human body. Next, processing one or more signals received from a plurality of sensors/electrodes configured in the sensor patch. Further, detecting a physiological signal from the processed signals and transmitting the detected physiological signal to a mobile device. The method also comprises providing power supply to the wireless monitoring device. The orientation of the sensor patch is detected using a plurality of sensors, wherein each of the plurality of sensors is one of accelerometer, gyroscope and magnetometer. The processing one or more signals, received from a plurality of sensors, comprises amplifying the one or more signals, filtering the amplified signals to filter out high frequency noise from the amplified sensed signals and converting the amplified sensed signals in to digital signal. The detected physiological signals are transmitted using at least one data transmission protocol selected from at least one of Bluetooth, Wireless fidelity (Wi-Fi), second generation (2G), third generation (3G), long term evolution (LTE) and any other wireless protocol.

The described operations may be implemented as a method, system or article of manufacture using standard programming and/or engineering techniques to produce software, firmware, hardware, or any combination thereof. The described operations may be implemented as code maintained in a “non-transitory computer readable medium”, where a processor may read and execute the code from the computer readable medium. The processor is at least one of a microprocessor and a processor capable of processing and executing the queries. A non-transitory computer readable medium may comprise media such as magnetic storage medium (e.g., hard disk drives, floppy disks, tape, etc.), optical storage (CD-ROMs, DVDs, optical disks, etc.), volatile and non-volatile memory devices (e.g., EEPROMs, ROMs, PROMs, RAMs, DRAMs, SRAMs, Flash Memory, firmware, programmable logic, etc.), etc. Further, non-transitory computer-readable media comprise all computer-readable media except for a transitory. The code implementing the described operations may further be implemented in hardware logic (e.g., an integrated circuit chip, Programmable Gate Array (PGA), Application Specific Integrated Circuit (ASIC), etc.).

Still further, the code implementing the described operations may be implemented in “transmission signals”, where transmission signals may propagate through space or through a transmission media, such as an optical fiber, copper wire, etc. The transmission signals in which the code or logic is encoded may further comprise a wireless signal, satellite transmission, radio waves, infrared signals, Bluetooth, etc. The transmission signals in which the code or logic is encoded is capable of being transmitted by a transmitting station and received by a receiving station, where the code or logic encoded in the transmission signal may be decoded and stored in hardware or a non-transitory computer readable medium at the receiving and transmitting stations or devices. An “article of manufacture” comprises non-transitory computer readable medium, hardware logic, and/or transmission signals in which code may be implemented. A device in which the code implementing the described embodiments of operations is encoded may comprise a computer readable medium or hardware logic. Of course, those skilled in the art will recognize that many modifications may be made to this configuration without departing from the scope of the invention, and that the article of manufacture may comprise suitable information bearing medium known in the art.

The terms “an embodiment”, “embodiment”, “embodiments”, “the embodiment”, “the embodiments”, “one or more embodiments”, “some embodiments”, and “one embodiment” mean “one or more (but not all) embodiments of the invention(s)” unless expressly specified otherwise.

The terms “including”, “comprising”, “having” and variations thereof mean “including but not limited to”, unless expressly specified otherwise.

The enumerated listing of items does not imply that any or all of the items are mutually exclusive, unless expressly specified otherwise.

The terms “a”, “an” and “the” mean “one or more”, unless expressly specified otherwise.

A description of an embodiment with several components in communication with each other does not imply that all such components are required. On the contrary a variety of optional components are described to illustrate the wide variety of possible embodiments of the invention.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the disclosure of the embodiments of the invention is intended to be illustrative, but not limiting, of the scope of the invention, which is set forth in the following claims.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

Referral Numerals

| Reference Number | Description |
|---|---|
| 100 | Wire-free electrode device |
| 102 | Auto-orientation module |
| 104 | Signal Processing Module |
| 106 | Detection Module |
| 108 | Compression Module |
| 110 | Data Transmission Module |
| 202 | Front end |
| 204 | Filter |
| 206 | Analog to Digital Converter (ADC) |

1. A wireless physiological device for acquiring and processing physiological signals, the device comprising:
a sensor patch comprising plurality of electrodes, configured to be in contact with a skin surface of a human body, to measure physiological of the patient; and
at least one wire-free module, embedded in the sensor patch, comprising
an auto-orientation module to detect the orientation of the sensor patch on the human body using a plurality of sensors;
a processing module to process one or more signals received from the plurality of electrodes;
a detection module to detect one or more processed signals as a predefined physiological data; and
a transmission module to transmit the detected physiological data to a mobile device.

2. The device as claimed in claim 1 further comprises a power source configured to supply power to the device.

3. The device as claimed in claim 1 further comprises a storage unit for storing at least one of signals sensed by the sensor patch, the processed signals and data detected physiological signals.

4. The device as claimed in claim 1 further comprises a compression module to compression the physiological signals to preserve in original form

5. The device as claimed in claim 1, wherein the auto-orientation module comprises plurality of sensors to detect the orientation of the sensor patch.

6. The device as claimed in claim 5, wherein each of the plurality of sensor is one of accelerometer, gyroscope and magnetometer.

7. The device as claimed in claim 1, wherein the processing module comprising:
front end module comprising at least one instrumentation block to amplify a plurality of signals sensed by the plurality of electrodes;
at least one filter to filter out noise from the amplified sensed signals; and
an analog to digital converter (ADC) to convert the amplified sensed signals in to digital signal.

8. The device as claimed in claim 1, wherein the transmission module uses at least one data transmission protocol selected from at least one of Bluetooth, Wireless fidelity (Wi-Fi), second generation (2G), third generation (3G), long term evolution (LTE) and any other wireless protocol.

9. The device as claimed in claim 1, wherein the transmission module comprises a data manager block to perform machine learning from the physiological data to obtain one of critical and non-critical event.

10. The device as claimed in claim 1, wherein the transmission module transmits physiological data using at least one of minimum available bandwidth data network for non-critical events and any available data network for critical events.

11. A method of acquiring and processing physiological signals using a wireless monitoring device, the method comprising:
identifying, by the wireless physiological device, orientation of a sensor patch placed on a human body;
processing, by the wireless physiological device, one or more signals received from a plurality of electrodes configured in the sensor patch;
detecting, by the wireless physiological device, a physiological data from the processed signals; and
transmitting, by the wireless physiological device, the detected physiological data to a mobile device.

12. The method as claimed in claim 11 further comprises providing power supply to the wireless monitoring device.

13. The method as claimed in claim 11 further comprises storing at least one of signals sensed by the sensor patch, the processed signals and data detected physiological signals, in a storage unit.

14. The method as claimed in claim 11 further comprises compressing the physiological signals to preserve in original form

15. The method as claimed in claim 11, wherein the orientation of the sensor patch is detected using a plurality of sensors, wherein each of the plurality of sensors is one of accelerometer, gyroscope and magnetometer.

16. The method as claimed in claim 11, wherein the processing one or more signals received from a plurality of electrodes comprising:
amplifying the one or more signals sensed by the plurality of electrodes;
filtering the amplified signals to filter out high frequency noise from the amplified sensed signals; and
converting the amplified sensed signals in to digital signal.

17. The method as claimed in claim 11, wherein the detected physiological signals are transmitted using at least one data transmission protocol selected from at least one of Bluetooth, Wireless fidelity (Wi-Fi), second generation (2G), third generation (3G), long term evolution (LTE) and any other wireless protocol.

18. The method as claimed in claim 11, wherein the transmission of physiological data further comprising performing machine learning from the physiological data to obtain one of critical and non-critical event.

19. The method as claimed in claim 11, wherein the transmission of physiological data using at least one of minimum available bandwidth data network for non-critical events and any available data network for critical events.
